(19) United States
(12) Patent Application Publication
(10) Pub. No.: US 2005/0015013 A1
(43) Pub. Date: Jan. 20, 2005

(54) Devices for stabilizing tissue

(75) Inventors: Alan J. Greszler, Elyria, OH (US); Daniel N. Kelsch, Fairview Park, OH (US)

Correspondence Address: PEARNE & GORDON LLP, 1801 EAST 9TH STREET, SUITE 1200, CLEVELAND, OH, 44114-3108, US

(73) Assignee: Biomec Inc., Cleveland, OH (US)

(21) Appl. No.: 10/867,089

(22) Filed: Jun. 14, 2004

Related U.S. Application Data
(60) Provisional application No. 60/478,160, filed on Jun. 13, 2003.

(51) Int. Cl. A61B 6/00; A61B 17/08

(52) U.S. Cl. 600/476; 606/151

(57) ABSTRACT

A device for stabilizing tissue includes a stabilization member with portions at least partially defining an observation area therebetween. The portions are adapted to contact tissue to stabilize tissue with respect to the stabilization member. The device further includes an apparatus attached with respect to the stabilization member. The apparatus includes at least one port adapted to emit light into the observation area and towards stabilized tissue.

### CROSS-REFERENCE TO RELATED APPLICATIONS

This application claims the benefit of U.S. Provisional Application No. 60/478,160, filed Jun. 13, 2003, hereby incorporated herein by reference.

### FIELD OF THE INVENTION

The present invention relates generally to stabilization devices for stabilizing tissue, and more particularly to stabilization devices adapted to emit light into an observation area and towards stabilized tissue.

### BACKGROUND OF THE INVENTION

Minimally invasive surgical procedures minimize the amount of trauma inflicted upon the patient during the procedure, and thereby minimize the amount of time required for the patient to recover. During such a procedure, an incision is often made that is large enough to provide the surgeon with suitable access to an observation area of surgical interest. During the surgical procedure, a surgeon and surgical assistants are typically located at convenient locations around the patient such that each individual has access to the observation area. While a small surgical team may be useful to complete a surgical procedure, one or more of the individuals might obstruct overhead lights positioned to illuminate the observation area. Therefore, the surgical procedure might be complicated by shadows obscuring the observation area. Furthermore, overhead lights may not be sufficient to illuminate certain observation areas located within the patient or obstructed by anatomical features of the patient.

Surgeons are known to wear a headlamp device in order to compensate for certain obstructions of overhead light that are typically caused by surgical team members, the location of the observation area, and/or anatomical features of the patient. A known headlamp device includes a harness for positioning the headlamp relative to the surgeon's head. The position of the headlamp is typically adjustable to allow the headlamp to emit light along the surgeon's line of sight to the observation area of surgical interest. While the headlamp device can facilitate hands-free illumination of an observation area during certain surgical procedures, movements of the surgeon's head can interrupt illumination of the observation area, thereby hampering the ability of the surgical assistants to continuously perform their duties. Further, the surgical operation might be interrupted due to fatigue, such as neck cramps, that might develop under the weight of the headlamp device.

In addition to illumination difficulties, certain surgical procedures require stabilization of tissue in order to perform delicate surgical procedures. For example, coronary artery bypass surgery traditionally requires stabilization of a surface of the heart in order to perform delicate surgical procedures on an obstructed coronary artery. It is known to stop the heart to bypass a section of a blocked, or partially blocked coronary artery that interferes with the delivery of oxygen to the heart. In such cases, a heart-lung machine is typically used to perform the functions of the heart and lungs during the surgical procedure. While the heart is stopped, a surgeon grafts an interior passageway to bypass the at least partially blocked portion of the coronary artery. The newly grafted interior passageway allows blood to pass freely therethrough, restoring the supply of oxygen to the heart. While stopping the heart is effective to stabilize the surgical area of interest, interrupting the heart function includes risks such as damage to the heart and surrounding tissue, exposure of the blood to machinery that can include contaminants, and failed attempts to restart the heart.

In order to avoid the risks associated with stopping the heart, it would be beneficial to perform the surgical procedure as the heart continues to function. However, a beating heart presents surgeons with a dynamic area of surgical interest, making it difficult to perform the delicate surgical procedure. Nearby surgical equipment can also inadvertently damage the heart due to movement of coronary tissue at the surgical area of interest.

To simplify the surgical procedure and prevent inadvertent damage to the heart, it is known to stabilize coronary tissue at the surgical area of interest while the heart continues to beat. To stabilize the coronary tissue, a surgeon typically uses a stabilizer that engages the area of surgical interest of the heart and minimizes the motion of that area. Friction, a vacuum, or a combination thereof are typically used by protruding arms of the stabilizer to engage portions of a surface of the heart on opposite sides of the area of surgical interest. With this area isolated and generally stabilized, the surgeon can graft the interior passageway to bypass the blocked coronary artery. However, illumination of the observation area of surgical interest continues to present a problem. Moreover, the protruding arms of the stabilizer might further obstruct light.

Accordingly, it would be beneficial to provide a device for stabilizing tissue that is adapted to emit light into the observation area and towards stabilized tissue.

### SUMMARY OF THE INVENTION

In accordance with one aspect, the present invention provides a device for stabilizing tissue. The device includes a stabilization member with portions at least partially defining an observation area therebetween. The portions are adapted to contact tissue to stabilize tissue with respect to the stabilization member. The device further comprises an apparatus attached with respect to the stabilization member. The apparatus includes at least one port adapted to emit light into the observation area and towards stabilized tissue.

In accordance with another aspect, the invention provides a device for stabilizing tissue. The device comprises a stabilization member adapted to contact tissue to stabilize tissue with respect to the stabilization member. The stabilization member comprises a first elongated foot and a second elongated foot that at least partially define an observation area therebetween. The device further comprises a first apparatus attached with respect to the first elongated foot and including at least one port adapted to emit light into the observation area and towards stabilized tissue.

In accordance with still another aspect, the present invention provides a device for stabilizing tissue. The device comprises a stabilization member adapted to contact tissue to stabilize tissue with respect to the stabilization member. The stabilization member comprises a first elongated foot and a second elongated foot extending substantially parallel with respect to one another and at least partially defining an observation area therebetween. The device further comprises a first apparatus attached with respect to the first elongated foot and including a first set of ports that are linearly offset from one another. The first set of ports are adapted to emit light into the observation area and towards stabilized tissue. The device also comprises a second apparatus attached with respect to the second elongated foot and including a second set of ports that are linearly offset from one another. The second set of ports are adapted to emit light into the observation area and towards stabilized tissue.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a perspective view of a device for stabilizing tissue in accordance with exemplary embodiments of the present invention;

FIG. 2A is a sectional view of the device for stabilizing tissue taken generally at section 2A-2A in FIG. 1;

FIG. 2B is a sectional view of a device for stabilizing tissue in accordance with further exemplary embodiments of the present invention;

FIG. 2C is a sectional view of a device for stabilizing tissue in accordance with still further exemplary embodiments of the present invention;

FIG. 2D is a sectional view of a device for stabilizing tissue in accordance with yet further exemplary embodiments of the present invention;

FIG. 3A is a sectional view of the device for stabilizing tissue taken generally at section 3A-3A in FIG. 1; and

FIG. 3B is a sectional view of a device for stabilizing tissue in accordance with additional exemplary embodiments of the present invention.

### DETAILED DESCRIPTION OF EXEMPLARY EMBODIMENTS

Certain terminology is used herein for convenience only and is not to be taken as a limitation on the present invention. Further, in the drawings, in order to clearly and concisely illustrate the present invention, certain features may be shown in somewhat schematic form.

Devices for stabilizing tissue are disclosed herein. Devices in accordance with the present invention include a stabilization member. A wide variety of stabilization members might be used in accordance with the present invention wherein such stabilization members are useful to facilitate stabilization of tissue. Exemplary stabilization members that might be used in accordance with the various exemplary embodiments herein are described in U.S. Pat. No. 5,727,569 to Benetti et al., U.S. Pat. No. 5,836,311 to Borst et al., U.S. Pat. No. 5,865,730 to Fox et al., U.S. Pat. No. 5,894,843 to Benetti et al., U.S. Pat. No. 6,019,722 to Spence et al. and U.S. Pat. No. 6,511,416 B1 to Green, II et al., which are each herein incorporated by reference.

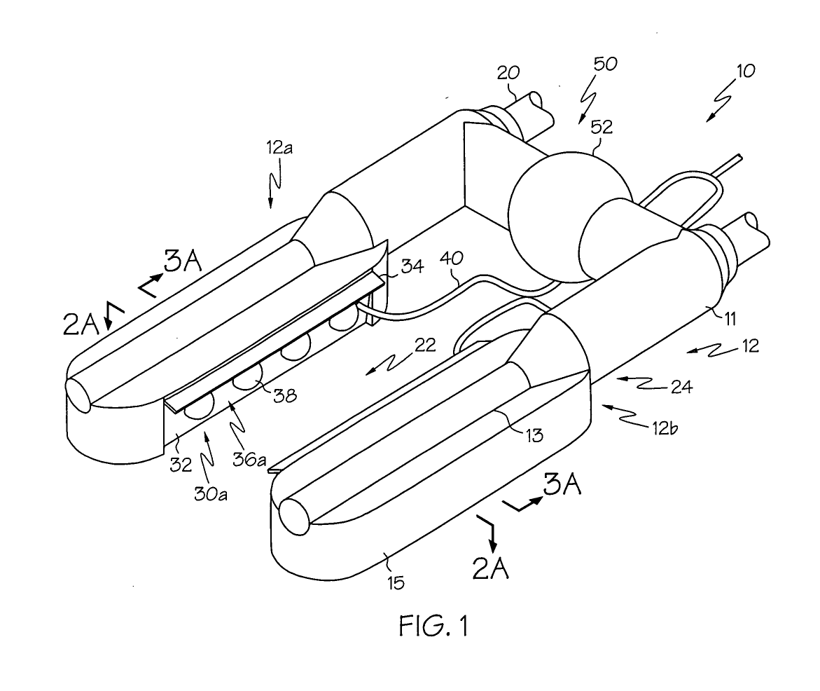

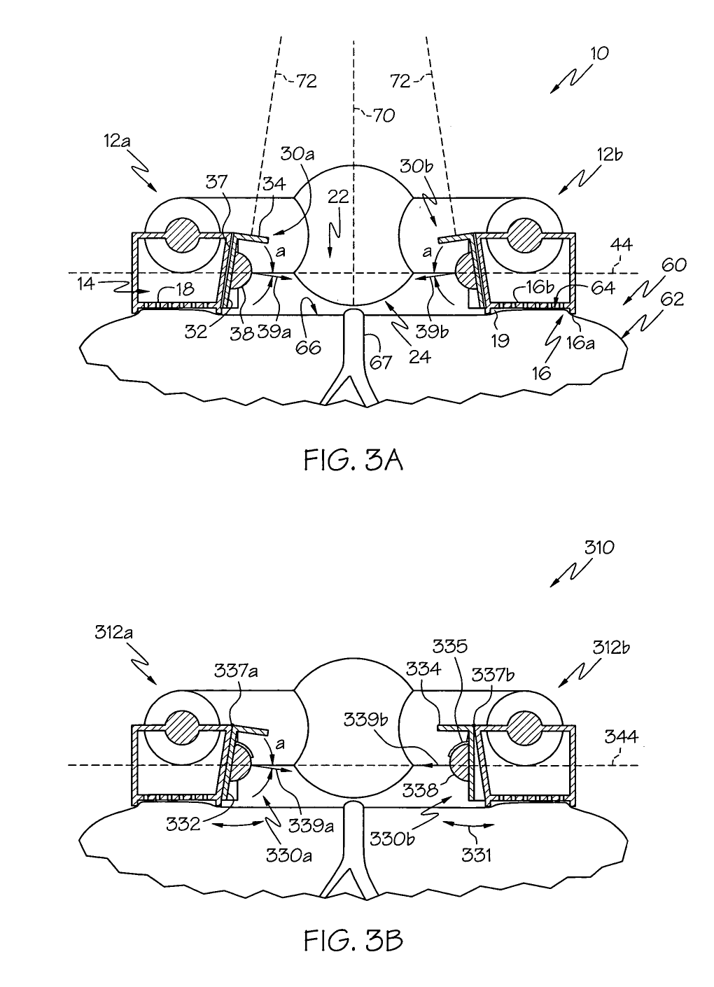

A device 10 including one particular exemplary embodiment of a stabilization member 12 is illustrated in FIG. 1. The illustrated stabilization member 12 includes portions that at least partially define an observation area 22 therebetween, wherein such portions are adapted to contact tissue to stabilize tissue with respect to the stabilization member 12. Such portions may comprise a wide variety of shapes and sizes to facilitate stabilization of tissue. In certain embodiments, the stabilization member might comprise an annular member, C-shaped member, or the like, with arcuate portions that define a stabilization area therebetween. At least one portion may also comprise an elongated foot. For example, as shown in illustrated exemplary embodiments, the portions of the stabilization member 12 can comprise a first elongated foot 12a and a second elongated foot 12b that at least partially define an observation area 22 therebetween. As shown in FIG. 3A, the first elongated foot 12a and the second elongated foot 12b optionally extend substantially along an extension plane 44 and, as shown, can be oriented substantially parallel with respect to one another.

As shown in FIG. 3A, each elongated foot 12a, 12b might comprise an engagement structure 16 adapted to contact a surface 64 of tissue 60 to be stabilized. In certain embodiments, the engagement structure might be adapted to gently grip the contact surface 64 of the tissue 60. As shown, the engagement structure 16 of each elongated foot might comprise a rib 16a and a wall 16b including apertures 18. The apertures 18 are in communication with a suction area 19 defined by the rib 16a and the wall 16b. Each elongated foot 12a, 12b includes an interior area 14 that can be placed in an underpressure condition by use of one or more vacuum tubes 20 that are connected to a conventional vacuum device to provide underpressurization to the interior area 14 of each elongated foot 12a, 12b. Underpressurization of the interior area 14 may be transmitted to the suction area 19 by way of one or more apertures 18. As shown in FIGS. 3A and 3B, placing the suction area 19 in an underpressure condition allows each elongated foot 12a, 12b to gently grip the tissue 60 by drawing portions of the contact surface 64 of the tissue 60 into the suction area 19. The gentle gripping of each elongated foot 12a, 12b presents a stabilized tissue surface 66 while other surfaces 62 of the tissue 60 are free to move. Similar exemplary engagement structures that might be incorporated with the concepts of the present invention are disclosed by U.S. Pat. No. 5,865,730 to Fox et al. and U.S. Pat. No. 6,511,416 to green, II et al., which are herein incorporated by reference.

Although not shown, the engagement structure 16 might comprise alternative arrangements to permit gripping of tissue. For example, the engagement structure might comprise one or more suction pods, flexible suction cups, clamps, fasteners, or the like. The engagement structure might also include a releasable adhesive layer. For example, a condition-sensitive adhesive, such as a polymorizable adhesive, might be employed that can be activated and/or deactivated by certain conditions such as wavelengths of light, levels of heat, chemicals or the like. Still further, the engagement structure might simply comprise a surface adapted to engage an area of the tissue wherein gripping of the tissue might not be necessary. Furthermore, pressing the surface with sufficient force against the contact surface of the tissue might provide a friction force sufficient to effectively grip the contact surface of the tissue. The coefficient of friction of the surface might also be increased to reduce the required pressure to achieve sufficient friction force.

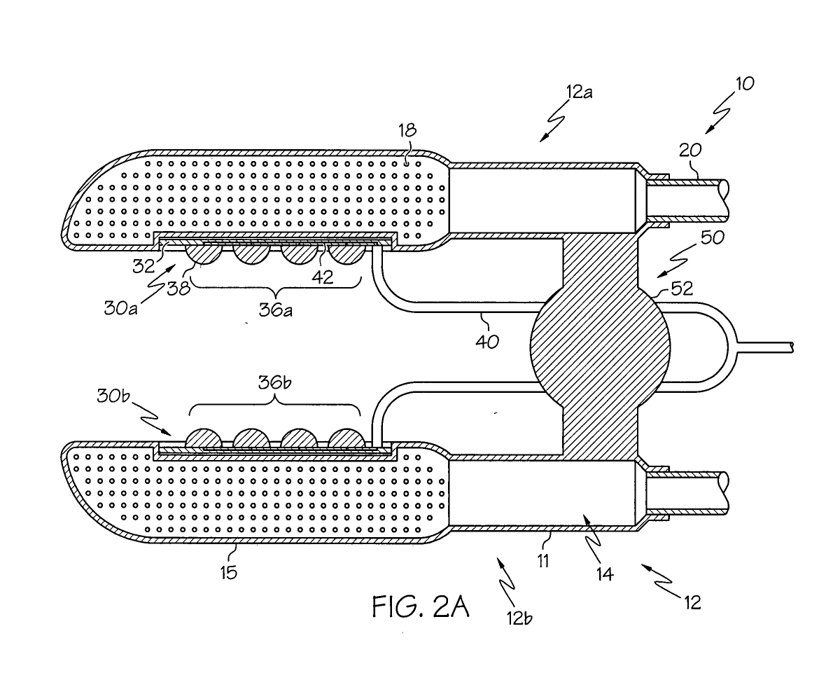

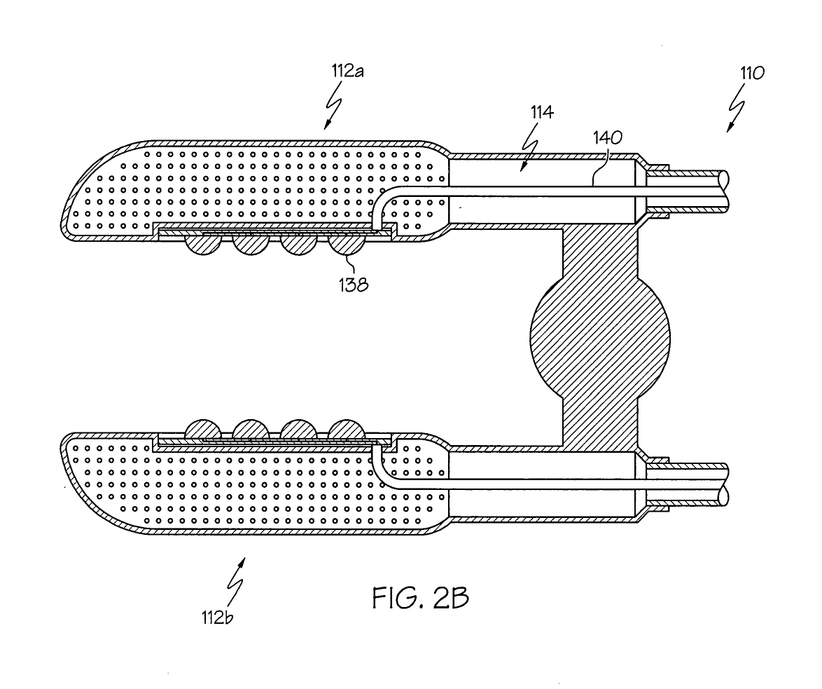

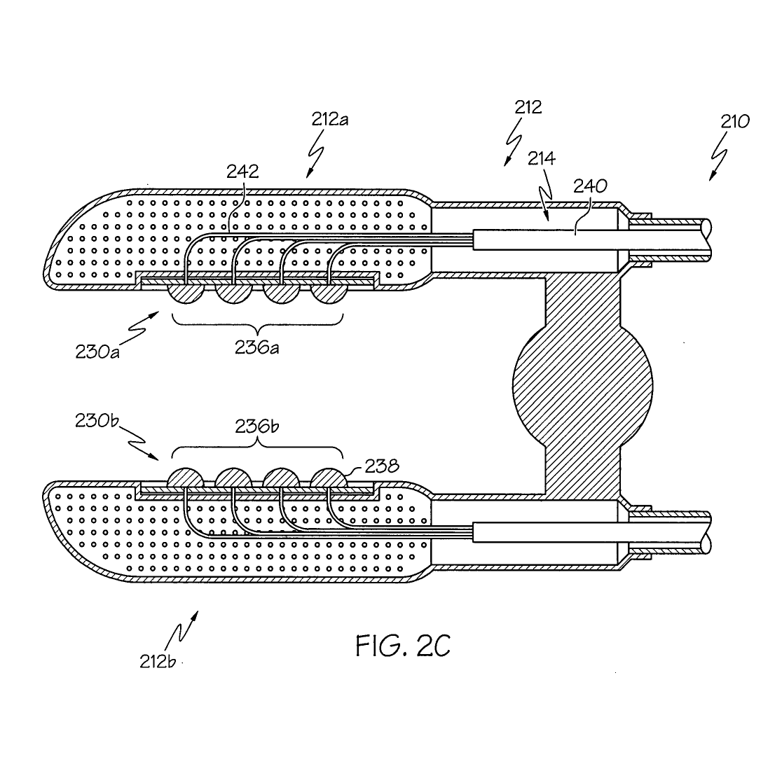

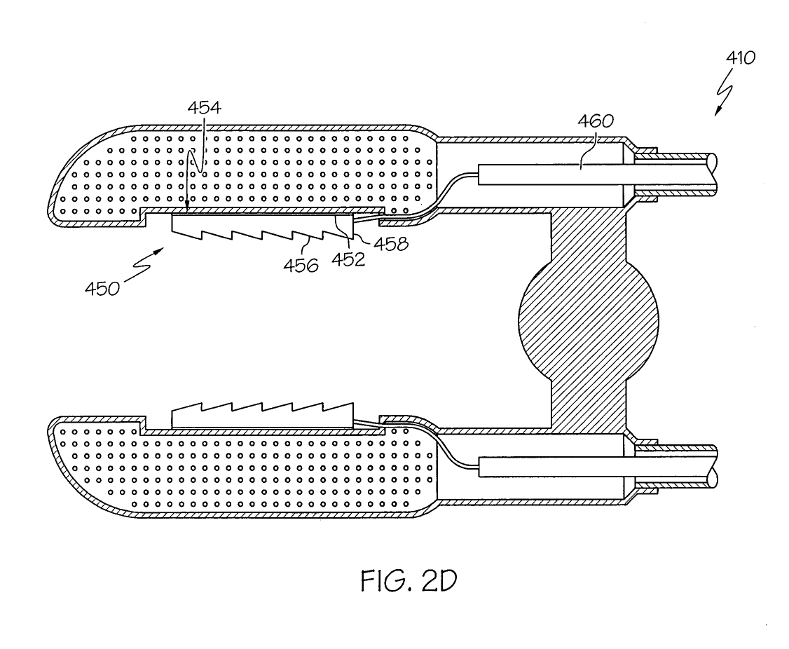

As further illustrated in the drawings, each elongated foot 12a, 12b might include a first portion 11 that is offset from a second portion 15 to provide an offset area 24. The offset area 24 prevents the first portion 11 of the stabilization member 12 from contacting tissue 60 as best illustrated in FIG. 3A. As shown in FIG. 2A, the interior area 14, if provided, can extend from the first portion 11 to the second portion 15. The shape of the interior area 14 is shown in general schematic form in order to demonstrate a fluid connection between the second portion 15 and the vacuum tube 20 by way of the interior area 14. The first portion 11 might also comprise an extension 13, such as a rod, to enhance the structural integrity of the second portion 15.

As further illustrated in FIG. 1, the stabilization member 12 can further include a cross member 50 extending between the first elongated foot 12a and the second elongated foot 12b. The cross member 50 provides a support structure for each elongated foot and might include a bulbous portion 52 to permit an articulate connection between a support arm (not shown) and the stabilization member 12. While each elongated foot 12a, 12b is shown rigidly connected to the cross member 50, further embodiments of the present invention might permit one or more elongated feet to be adjustably connected to the cross member. For example each elongated foot may be rotatably mounted with respect to the cross member to permit each elongated foot to rotate with respect to the cross member about its elongated axis. In additional embodiments, each elongated foot may be pivotally mounted with respect to the cross member to permit an angular adjustment between the cross member and the elongated foot. For example, pivotal mounting of at least one of a pair of elongated feet would permit alternative mounting arrangements where the elongated feet are not substantially parallel with respect to one another.

Each device of the present invention further includes an apparatus attached with respect to the stabilization member that includes at least one port adapted to emit light into the observation area and towards the stabilized tissue. Emitting light from the device is desirable to illuminate a stabilized tissue surface to facilitate observation. In accordance with the present invention, the port might be adapted to emit light by including a light-producing source. For example, the port might comprise an electrical illumination source such as a light-emitting diode as illustrated in FIGS. 1 and 2A. Other electrical illumination sources might comprise miniature incandescent lights, flourescent lights, or the like. Moreover, other exemplary light-producing ports might comprise naturally illuminant materials, materials that retain light energy for a subsequent gradual release, or the like, or any other source of light. In further exemplary embodiments, the port might be adapted to emit light by including structure that permits passage of light. For example, the port might include a lens, a portion of a fiber optic line, a surface location, an opening, or the like. Furthermore, a port may comprise various sizes and shapes and can comprise a single point location, a plurality of locations with various alternative patterns, an area such as a geometric shape, elongated shape or the like.

To maximize illumination, the at least one port might comprise a plurality of ports that are offset from one another. As shown, for example, a first set of ports 36a may be provided that are offset from a second set of ports 36b. The first and second set of ports 36a, 36b each further include a plurality of ports 38 that are linearly offset from one another. Providing a linear offset allows substantially consistent and continuous illumination of the observation area 22 along substantially the entire length of the observation area while minimizing the number of ports necessary to provide such consistent and continuous illumination. Providing a linear offset is particularly useful for observation of an elongated vessel, such as a coronary vessel 67 of a heart, that is stabilized and straddled by the elongated feet. A plurality of ports having additional offset patterns might also be employed depending on the particular application. For example, certain ports from a set of ports might have a smaller offset (and therefore have a greater port density) in areas where enhanced illumination is desired.

The at least one port of the present invention might also be adapted to alter light in order to provide enhanced observation, such as enhanced contrast or illumination, of stabilized tissue. For instance, the at least one port might be adapted to alter the diffusing pattern of the light, alter the wavelength of the light, and/or filter predetermined wavelengths of light. In exemplary embodiments, the at least one port might comprise a lens with different geometries adapted to alter light and/or a colored, transparent, translucent and/or diffusing lens adapted to alter light. The port may also include a filter device adapted to alter light. The filter device, if provided, might comprise a removable filter to allow alternative light selection. Therefore, exemplary ports in accordance with the present invention may emit light in a wide variety of ways and may emit white light and/or other light colors with a wide spectrum of alternative light wavelengths.

As described above, the at least one port in accordance with various embodiments of the present invention might include a port requiring electricity to function, such as an electrical illumination source. A light-emitting diode, for example, might require a source of electricity to allow the diode to produce light. In order to provide a port with electricity, the device may comprise a wire adapted to transmit electricity to the at least one port. The wire can provide electricity from a source of energy that will operate the light-emitting diode or other electrically operated port. The source of energy can comprise a battery, or other source of electricity and the wire can comprise a portion of an electrical circuit to operate the electrically operated port.

As described above with reference to FIGS. 1 and 2A above, for example, the wire 40 is adapted to provide the port 38 with electricity from a source of energy (not shown). As shown in FIGS. 1 and 2A, the wire might be located external to the first elongated foot 12a and the second elongated foot 12b. Providing the wire outside the elongated feet might simplify fabrication of a device and/or might simplify retrofit of an existing stabilizer to include an apparatus adapted to emit light. In alternative embodiments, the wire might be sheltered by outer portions of the device. For example, as shown in FIG. 2B, a device 110 might include a stabilization member 110 including a first elongated foot 112a and a second elongated foot 112b that each include an interior area 114. As shown, at least one wire 140 at least partially extends within the interior area 114 and is adapted to transmit electricity to at least one port 138 (e.g., a light-emitting diode). The interior area, for example, might comprise the interior area for underpressurization in embodiments using underpressurization to permit the device to grip a contact surface of the tissue. Locating the wire within portions of the device prevents potential interference with the surgical procedure, protects the wire and prevents tangling or snagging of the wire with foreign objects.

FIGS. 1 and 2A depict details of a first apparatus 30a and a second apparatus 30b that each comprise a plurality of ports 38 including a plurality of light-emitting diodes that are offset from one another. A schematic depiction of a port comprising a light-emitting diode is illustrated in FIG. 2A wherein a wire 40 may be provided and electrically connected with the light-emitting diodes of a first set of diodes 36a and with the light-emitting diodes of a second set of diodes 36b by way of a corresponding electrical connection 42 provided with each apparatus 30a, 30b. In the illustrated embodiment, each apparatus 30a, 30b includes a base member 32 adapted to mount the light-emitting diodes and support the electrical connection 42. As shown, the electrical connection 42 might be embedded within the base member 32. In alternative embodiments the electrical connection is provided on a back surface of the base member 32 wherein the attachment between the base member 32 and the corresponding elongated foot provides sufficient isolation of the electrical connection 42 from external locations of the apparatus.

Each apparatus of the present invention may be attached with respect to the stabilization member in a variety of ways. Each apparatus, for example, might be indirectly attached to the stabilization member by an intermediate member, such as a bracket or extension (not shown). Each apparatus might also be directly attached to the stabilization member. As shown in FIG. 3A, for example, the base 32 of each apparatus may be attached to a surface of the stabilization member 12 with an adhesive layer 37. The adhesive layer might provide a seal sufficient to prevent transfer of bodily fluids and matter from entering between the base member 32 of the stabilization member 12. Isolation of the electrical components is desirable to minimize the possibility of electrical malfunction of the device and/or electric shock to the patient. The seal also minimizes the likelihood that the area of surgical interest will be contaminated by the device. Providing an adhesive layer may also be beneficial to attach one or more apparatus to retrofit an existing stabilizer for forming a device in accordance with the present invention.

In addition to an adhesive layer, apparatus in accordance with the present invention might be attached to the stabilization member in a large variety of ways. For example, apparatus might be attached to the stabilization member with mechanical fasteners or the like. Apparatus might also be integrally attached to the stabilization member by way of sonic welding, chemical welding, or integrally forming the stabilization member and the apparatus as one-piece member.

Attachment of the apparatus with respect to the stabilization member might comprise a stationary attachment such that the stabilization member and the apparatus are nonadjustably attached to one another. In addition, each apparatus may be attached with respect to the stabilization member at various angles. For example, as shown in FIG. 3A, the first apparatus 30a is attached such that a corresponding port 38 faces a first direction 39a extending towards the observation area 22 and at an angle “a” with respect to the extension plane 44. Similarly, the second apparatus 30b is attached such that a corresponding port 38 faces a second direction 39b extending towards the observation area 22 at an angle “a” with respect to the extension plane 44. Facing the corresponding ports towards the observation area 22 and at an angle “a” with respect to the extension plane 44 can facilitate illumination of the stabilized tissue surface 66.

In alternative embodiments, attachment of at least one apparatus with respect to the stabilization member might comprise an adjustable attachment such that the stabilization member and the apparatus are adjustably attached to one another. For example, as shown in FIG. 3B, a device 310 includes a first apparatus 330a that is pivotally attached to a first elongated foot 312a for pivotable adjustment of the first apparatus 330a with respect to the first elongated foot 312a about a first pivot axis 337a. Similarly a second apparatus 330b is pivotally attached to a second elongated foot 312b for pivotable adjustment of the second apparatus 330b with respect to the second elongated foot 312b about a second pivot axis 337b. Accordingly, one or both of the apparatus might be adjustable to various angles. As shown in FIG. 3B, for example, the first apparatus 330a might be include a port 338 facing a first direction 339a extending towards the observation area and at an angle “a” with respect to an extension plane 344. The second apparatus 330b is shown at an alternative angular orientation wherein the second apparatus 330b has been pivoted outward along pivot arrow 331 about the pivot axis 337b to a position wherein a corresponding port 338 faces a second direction 339b parallel with the extension plane 344. Allowing adjustment, such as angular adjustment, of the apparatus with respect to the stabilization device permits illumination of tissues having various alternative stabilized tissue surface characteristics.

As discussed previously, each port in accordance with the present might be adapted to emit light by including a light-producing source, such as a light-emitting diode. In alternative exemplary embodiments, the port might be adapted to emit light by including structure that permits passage of light. For example, while various structures might be provided, FIG. 2C depicts an exemplary device 210 including a first apparatus 230a associated with the first elongated foot 212a and a second apparatus 230b associated with the second elongated foot 212b. The first apparatus 230a includes a first set of ports 236a and the second apparatus 230b includes a second set of ports 236b. Each set of ports 236a, 236b include a plurality of ports 238 offset from one another. Each of the plurality of ports 238 are adapted to permit passage of light. As shown, for example, each port 238 might comprise a lens structure adapted to emit light received from an optic fiber line 242. Use of a fiber optic line prevents potential electrical shock to the patient and might also provide an increased level of light intensity that might not be available or difficult to achieve with an electrical light-emitting diode.

The optic fiber line 242, if provided, can comprise a single optical fiber or might comprise a bundle of fibers to provide adequate transmission of light from a source of light (not shown) to each individual port 238. In exemplary embodiments, each port 238 includes a corresponding distinct optical fiber line 242 to allow control of light being emitted from each individual port. Providing distinct optical fiber lines 242 may provide beneficial illumination characteristics by allowing individual and customized illumination from each port. For example, the source of light may be designed to provide each port 238 with varying light intensities to illuminate one portion of stabilized tissue differently than another portion of stabilized tissue. In addition, the type of light (e.g., light color) might be varied between the ports to provide appropriate contrast and/or observation of different surface characteristics of the stabilized tissue.

It is contemplated that a single fiber optic line, including a single or grouping of optical fibers, might provide simultaneous illumination of each port of one or both sets of ports. For example, a single grouping of optical fibers might communicate simultaneously with the first set of ports to provide illumination of each port of the first set of ports simultaneously.

FIG. 2D depicts one particular embodiment of a device 410 that might incorporate a single optic line, including a single or grouping of optical fibers. FIG. 2D depicts a schematic cross sectional view of an exemplary light emitting panel 450 that can include light-reflecting features on the front and/or back surfaces of the panel. For example, a reflector 452 may be incorporated on a back side 454 of the light emitting panel 450. Light entering at a location of the light emitting panel may be reflected by the reflector 452 and out through each of the plurality of light-emitting lenses 456. In the illustrated embodiment, light is provided to an end 458 of the light emitting panel by a fiber optic line 460 comprising a single or grouping of optical fibers. In alternative embodiments, light can be introduced by any light producing source. For example, an electrical illumination source, such as a light-emitting diode, or the like, may be incorporated at the end of the light emitting panel.

One or more fiber optic lines might also extend through an interior area of the stabilization member or might extend external to the stabilization member. As further shown in FIG. 2C, for example, a fiber optic line 240 may carry each fiber optic line 242 through an interior area 214 of the stabilization member 212. Locating the fiber optic line within portions of the stabilization device prevents potential interference with the surgical procedure, protects the fiber optic line and prevents tangling or snagging of the fiber optic line with foreign objects.

As described above, ports of the present invention might comprise a wide variety of shapes and sizes. As shown in the exemplary embodiments herein, ports might comprise a hemispherical dome shape. With respect to FIGS. 1 and 2A, for example, the port might comprise a hemispherical dome shaped light-emitting diode. With respect to FIG. 2C, the port might alternatively comprise a hemispherical dome shaped lens. The hemispherical dome shape of the port can be adapted to enhance light dispersion to suitably illuminate the stabilized tissue surface 66. For example, the hemispherical dome shaped lens of the port 238 illustrated in FIG. 2C may be adapted to alter light by diffusing the light through the lens. While dispersing light in a hemispherical direction might be beneficial to maximize illumination of the stabilized tissue, light might unfortunately be directed straight along a line of sight of an observer. For example, as shown in FIG. 3A, light might be emitted from the port 38 in a direction along the line of sight 72 of an observer. In order to minimize or prevent light from being directly emitted to the eyes of an observer, the device might comprise an optional blind adapted to shield light emitted from the port in a direction away from the stabilized tissue surface 66 along the line of sight 72 of an observer. The blinds also permit viewing of light reflected from the stabilized tissue surface 66, for example, along a line of sight 70 of an observer. As shown in FIG. 3A, one exemplary embodiment includes a structure with a base member 32 and a blind comprising a flange 34 extending away from the base member 32.

FIG. 3B depicts another example of a blind comprising a flange 334 extending away from a base member 332. In addition, or alternatively, the port might incorporate a blind for shielding a portion of the light emitted from the port. For example, as shown in FIG. 3B, the port 338 might include a blind 335, such as a coating of opaque material, on an upper portion of the port 338 to minimize or prevent light from being emitted along the line of sight directly to the eyes of an observer. While the port depicts a coating 335 on the exterior surface of the port, it is understood that the coating might exist on the interior surface of the port. Moreover, the blind might comprise other structures and can include, for example, differing material types of the port wherein light may be emitted from a transparent material at desired locations of the port and blocked by an opaque material at other desired locations of the port.

Although not illustrated, ports of the present invention might include shapes other than a hemispherical dome shaped port. For example, ports might comprise a flat or convex shape. In addition, ports of the present invention might not extend outwardly from the surface of the base member. For example, the ports might comprise a structure that is flush or recessed within the base member of the apparatus.

Although devices herein have been described as beneficial for use in stabilizing tissue of a beating heart, devices herein may be adapted to stabilize an area of various anatomical features of a patient such as muscle, organ or other tissue. Moreover, from the above description of the invention, those skilled in the art will perceive improvements, changes and modifications. Such improvements, changes and modifications within the skill of the art are intended to be covered by the appended claims.

1. A device for stabilizing tissue comprising:
a stabilization member including portions at least partially defining an observation area therebetween, wherein the portions are adapted to contact tissue to stabilize tissue with respect to the stabilization member; and
an apparatus attached with respect to the stabilization member, the apparatus including at least one port adapted to emit light into the observation area and towards stabilized tissue.

2. The device of claim 1, wherein the at least one port comprises a plurality of ports that are offset from one another.

3. The device of claim 2, wherein the plurality of ports include a set of ports that are linearly offset from one another.

4. The device of claim 2, wherein each of the plurality of ports is provided with at least one fiber optic line for transmitting light to the corresponding port.

5. The device of claim 1, wherein the apparatus is provided with at least one wire adapted to transmit electricity to the at least one port.

6. The device of claim 1, wherein the stabilization member includes an interior area and the apparatus is provided with at least one wire at least partially extending within the interior area and adapted to transmit electricity to the at least one port.

7. The device of claim 1, wherein the at least one port includes an electrical illumination source.

8. The device of claim 7, wherein the electrical illumination source includes a light-emitting diode.

9. The device of claim 1, wherein the at least one port is provided with at least one fiber optic line for transmitting light to the at least one port.

10. The device of claim 1, wherein the at least one port comprises a light emitting panel.

11. The device of claim 1, wherein the stabilization member includes an interior area and the apparatus is provided with at least one fiber optic line at least partially extending within the interior area and adapted to transmit light to the at least one port.

12. The device of claim 1, wherein the apparatus is adjustably attached with respect to the stabilization member to permit selective positioning of the apparatus and the stabilization member to one of a plurality of alternative angular positions with respect to one another.

13. The device of claim 1, further comprising a blind adapted to shield light emitted from the at least one port in a direction away from stabilized tissue.

14. The device of claim 1, wherein the apparatus includes a structure comprising a base member including the at least one port, and the structure further comprising a blind adapted to shield light emitted from the at least one port in a direction away from stabilized tissue.

15. The device of claim 14, wherein the blind comprises a flange extending away from the base member.

16. The device of claim 1, wherein the at least one port is adapted to alter light.

17. A device for stabilizing tissue comprising:
a stabilization member adapted to contact tissue to stabilize tissue with respect to the stabilization member, the stabilization member comprising a first elongated foot and a second elongated foot that at least partially define an observation area therebetween;
a first apparatus attached with respect to the first elongated foot and including at least one port adapted to emit light into the observation area and towards stabilized tissue.

18. The device of claim 17, further comprising a second apparatus attached with respect to the second elongated foot including at least one port adapted to direct light into the observation area and towards stabilized tissue.

19. The device of claim 17, wherein the first elongated foot and the second elongated foot extend substantially along an extension plane.

20. The device of claim 19, wherein the at least one port of the first apparatus faces a direction extending towards the observation area and at an angle with respect to the extension plane.

21. A device for stabilizing tissue comprising:
a stabilization member adapted to contact tissue to stabilize tissue with respect to the stabilization member, the stabilization member comprising a first elongated foot and a second elongated foot extending substantially parallel with respect to one another and at least partially defining an observation area therebetween;
a first apparatus attached with respect to the first elongated foot and including a first set of ports that are linearly offset from one another, wherein the first set of ports are adapted to emit light into the observation area and towards stabilized tissue; and
a second apparatus attached with respect to the second elongated foot and including a second set of ports that are linearly offset from one another, wherein the second set of ports are adapted to emit light into the observation area and towards stabilized tissue.

22. The device of claim 21, wherein the stabilization member further comprises a cross member extending between the first elongated foot and the second elongated foot.

23. The device of claim 21, wherein the first elongated foot and the second elongated foot extend substantially along an extension plane, the first set of ports each face a first direction extending towards the observation area and at an angle with respect to the extension plane, and the second set of ports each face a second direction extending towards the observation area and at an angle with respect to the extension plane.
